(19) United States
(12) Patent Application Publication
(10) Pub. No.: US 2016/0374624 A1
(43) Pub. Date: Dec. 29, 2016

(54) SYSTEMS AND METHODS FOR NONINVASIVE INTRACRANIAL PRESSURE CALIBRATION WITHOUT THE NEED FOR INVASIVE ICP

(71) Applicant: THE REGENTS OF THE UNIVERSITY OF CALIFORNIA, Oakland, CA (US)

(72) Inventor: Xiao Hu, San Francisco, CA (US)

(73) Assignee: THE REGENTS OF THE UNIVERSITY OF CALIFORNIA, Oakland, CA (US)

(21) Appl. No.: 15/259,305

(22) Filed: Sep. 8, 2016

Related U.S. Application Data
(62) Division of application No. 14/079,618, filed on Nov. 13, 2013.

(51) Int. Cl. A61B 5/00; A61B 5/026; A61B 5/03; A61B 5/0205

(52) U.S. Cl. CPC A61B 5/7278; A61B 5/0205; A61B 5/7267; A61B 5/026; A61B 5/031; A61B 5/7225; A61B 5/021

(57) ABSTRACT

Systems and methods for assessing a non-accessible parameter from accessible parameters are provided. A training process produces three databases: A dynamical model database containing input/output (I/O) models relating ABP/CBFV to ICP when estimating ICP; a mapping function database containing a mapping function for each entry in the model database, providing estimated of the dissimilarity between the unknown ICP and simulated ICP using the corresponding dynamical model on a given instance of ABP/CBFV; and a query feature database of vectors extracted from an instance of ABP/CBFV. New ABP/CBFV measurements are analyzed to extract query features that are evaluated by each mapping function in the database. The output is dissimilarity metrics providing estimates of the quality of the simulated ICP using the database models for a given ABP/CBFV instance. The dissimilarity metrics are ranked to find the optimal model. The model is used to simulate ICP using the new ABP/CBFV.

### CROSS-REFERENCE TO RELATED APPLICATIONS

This application is a division of U.S. patent application Ser. No. 14/079,618 filed on Nov. 13, 2013, incorporated herein by reference in its entirety.

### STATEMENT REGARDING FEDERALLY SPONSORED RESEARCH OR DEVELOPMENT

This invention was made with Government Support under NS066008, awarded by the National Institutes of Health. The Government has certain rights in the invention.

### INCORPORATION-BY-REFERENCE OF COMPUTER PROGRAM APPENDIX

Not Applicable

### BACKGROUND OF THE INVENTION

1. Field of Invention

This invention pertains generally to methods for processing sensed physiological information and more particularly to a system and method for non-invasive Intracranial Pressure Pulse (ICP) signal analysis and tracking of pulse metrics for real time diagnosis and prospective treatments.

2. Background

The treatment of many neurological disorders and brain injuries relies on the continuous measurement of different physiological signals like Electrocardiogram (ECG), Intracranial Pressure (ICP), Saturation of Peripheral Oxygen (Spo2), and Arterial Blood Pressure (ABP) by physicians. Dynamic changes in the intracranial pressure (ICP) reflect the ability of the body to compensate for changes in volume within the skull and pathophysiological changes in the cerebral vasculature. ICP monitoring is an essential element of current treatment protocols.

The conventional method for assessing ICP is with the surgical insertion of a catheter into one of the lateral ventricles of the brain of the patient that is then connected to an external pressure transducer. The output of the transducer produces a characteristic ICP pulse waveform. It has been recognized that an ICP pulse is typically triphasic with three subpeaks that originate mostly from cerebral arterial pulsations with some contributions of venous origin.

Although intracranial pressure (ICP) is an important physiological variable for managing brain injury patients, no clinically accepted medical device exists for noninvasive ICP assessment. Existing efforts to make a viable noninvasive ICP device have been focused on finding noninvasive alternative signals to correlate with ICP but their achievable accuracy is limited due to a lack of an individualized calibration module.

A logical attempt to create noninvasive ICP technology has been to infer ICP from other available physiological signals. Many such attempts in the past have focused on creating noninvasive sensors to measure ICP-related signals. Examples of such efforts include noninvasive ICP surrogates from brain imaging, pupillary size, presence of papilledema, optic nerve sheath width, retinal vein outflow pressure, visual evoked potentials, displacement of the skull, brain tissue resonance, cerebrospinal fluid (CSF) flow and CBF as assessed by phase-contrast MRI, CBFV as measured by Transcranial Doppler, and displacement of tympanic membrane.

A common scientific challenge to these existing efforts is to build in vivo calibration to obtain absolute ICP from its related signals. Without calibration, only ICP trending can be potentially inferred without any clue to the absolute ICP. Nevertheless, in-vivo noninvasive calibration is not a trivial problem. A global equation will fail because individual patient entails different equations to obtain accurate result. On the other hand, a straightforward regression to build a calibration equation for each patient is not feasible because the ICP is not readily obtainable noninvasively for each new patient as a starting point. In other words, the calibration problem is challenging because no global equation exists to transform values of an ICP-related signal to absolute value of ICP because individual subject entails different calibration functions that cannot be pre-built without having access to invasive ICP for the specific subject.

Despite the importance of ICP monitoring, signal processing capabilities in existing commercial ICP monitoring devices remain poor providing clinicians with a limited amount of information and reliance on invasive procedures. As a consequence, clinical decisions related to treating ICP-related abnormalities are typically made solely based on mean ICP.

Accordingly, there is a need for a system and method for non-invasive ICP monitoring from ICP-related signals to provide an accurate ICP estimation. There is also a need for a system and method for ICP monitoring that not only evaluates mean ICP, but can continuously evaluate the dynamic morphological features of the ICP pulse wave forms that is at the same time accurate, reliable and computationally practical. The present methods satisfy these needs, as well as others, and are generally an improvement over the art.

### SUMMARY OF THE INVENTION

The present invention provides an approach for achieving individualized calibration without using invasive ICP for a de novo patient. Generally the method is built upon the integration with system identification and a case based reasoning framework. A core element in this framework is a mapping function associated with an input/output model used to simulate ICP for a given noninvasive signal. This mapping function maps a noninvasive feature vector to an expected error of estimating ICP using the input/output model and hence facilitates the determination of an optimal model from a collection of models. The various solutions to learning the mapping functions are illustrated with a baseline multivariate linear model, a model with ranking constraints, and a kernelized model.

The method is a Noninvasive ICP Calibration Framework (NICF) consisting of two key elements: dynamical system modeling and case-based reasoning. The methods identify individual input/output models between invasive ICP and noninvasive signals from each subject of a training population. To obtain noninvasive ICP for a new patient, an optimal dynamical model is selected from this collection of models and used to simulate ICP from the noninvasive signals. System identification is used to identify dynamical models from the training data in the database. These models can simulate ICP using noninvasive signals as input. Then a case-based reasoning process is used on noninvasive signals of a new subject to locate the optimal ICP simulation model from the database that are estimated to be able to provide the best possible estimate of ICP for that patient.

Finding the optimal model using noninvasive signals is essentially a ranking process. The algorithm leverages this insight by incorporating ranking constraints when learning the functions that map noninvasive signals to a quality measure of the ICP estimate. Therefore, it can deliver a much more accurate estimation of ICP than other approaches.

The learning algorithm for the mapping function involves a sequential convex optimization of a regularized objective function with ranking constraints. Imposing ranking constraints is vital because the correct ranking of dynamic models based on mapping function output for a given query needs to be ensured to achieve accurate retrieval of the model. Furthermore, a kernelized version of this learning algorithm was derived to enable nonlinear mapping. The nonlinear mapping function, learned using this constraint learning algorithm, performed the best in the validation studies achieving a performance equivalent to a hypothetical case retrieval method that returns consistently the best 30th model to each query.

The system and method will not only provide end-users an ICP estimate but also a quality indicator of the reliability of the estimate. The method also guarantees an individualized calibration function.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 is a schematic block diagram of a system framework for estimating inaccessible biological variables from new accessible biological signal data according to one embodiment of the invention.

FIG. 2A is a system diagram model where ICP is an output from a dynamical system driven by its related noninvasive signals.

FIG. 2B is a system diagram model where ICP is an input to drive a dynamical system from which ICP-related noninvasive signals are observed.

FIG. 2C is a system diagram model where ICP is among the observables of a dynamical system that also include its related noninvasive signals.

FIG. 3 is a schematic block diagram of the NICP calibration framework (NICF) process according to one embodiment of the invention.

FIG. 4 is a flow diagram of the noninvasive ICP calibration framework optimal model selection process algorithm according to one embodiment of the invention.

### DETAILED DESCRIPTION OF THE INVENTION

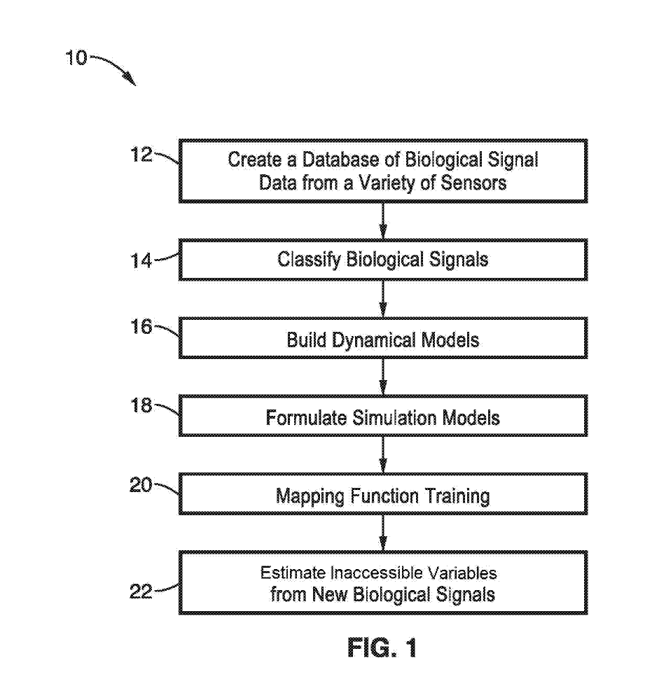

Referring more specifically to the drawings, for illustrative purposes several embodiments of the system and methods for individualized noninvasive intracranial pressure calibration of the present invention are depicted generally in FIG. 1 through FIG. 4. It will be appreciated that the methods may vary as to the specific steps and sequence and the system elements may vary as to structural details, without departing from the basic concepts as disclosed herein. The method steps are merely exemplary of the order that these steps may occur. The steps may occur in any order that is desired, such that it still performs the goals of the claimed invention.

The framework 10 shown in FIG. 1 is centered on trained mapping functions including a baseline multivariate linear model, a model with ranking constraints or a kernelized regression model. Generally the implementation of the method can be logically separated into a training phase and a running phase. For the training phase, a database is constructed, variable features are derived, and system identification is conducted along with the training of mapping functions. The training phase can be computationally demanding and time consuming to decide key system parameters, such as which feature or mapping function to use.

Once the system is trained, the development of a running system will be less involved in terms of algorithms because all the components required for online running should have already been developed in the training phase.

At block 12, a database of biological or physiological signal data from a plurality of sensors and a variety of sources is created. The nature of the biological signal data will be determined by the type of system that is investigated.

For example, one physiological parameter may include invasive intracranial pressure (ICP), the second physiological parameter may include cerebral blood flow velocity (CBFV), and a third physiological parameter may include arterial blood pressure (ABP) data. The signal database can store other relevant signals, such as PCO₂, ECG, arterial PO₂, etc. The length of each entry into the signal database is preferably long enough to facilitate the extraction of low frequency information and short enough to be reasonably treated as coming from a dynamic system with constant parameters.

While the embodiments of the systems and methods disclosed herein relate to estimating ICP values through noninvasive procedures, these methods and systems can be used in determining other conditions, and any other random variable may be estimated using these systems and methods. For example, risks of disease or mortality, or other physiological parameters than ICP, particularly those that vary with respect to time, can be estimated through the systems and methods disclosed herein.

Additionally, ABP and CBFV are used as exemplary physiological parameters from which biological signal data is obtained. However, ABP and CBFV are provided as only examples and other physiological parameters can be measured and utilized in accordance with the systems and methods described herein.

At block 14, the acquired biological signals from the sensor sources are optionally classified as system variables according to dynamical relationships and parameters. For example in a time series estimation system, target time series (TTS) and its related time series (RTS) can be classified in different sets of observed variables from the same dynamic system. TTS and RTS may be used as the output and input of a time series estimation process to derive variables less accessible from a set of related and more accessible variables in this illustration.

In another embodiment, each entry in the signal database can contain two types of signal classifications, desired signals and related signals. For example, invasively measured ICP can be a desired signal and other variables, including ABP and CBFY, are classified as related signals.

Dynamical models are built at block 16 that potentially formalize the relationship between feature variables in the database. Models are preferably developed and cross-validated. One operating principle of the method is to first build a collection of dynamical models and then use an instance of ICP-related signals to locate the optimal models within the collection to simulate ICP for this instance. For example, potential models of the association between RTS and TTS can be categorized into three types: 1) RTS is an input signal to the dynamic system that generates TTS; 2) TTS is an input signal to the dynamic system that generates RTS; and 3) RTS and TTS are both generated by a dynamic system.

Given this categorization, the third scenario, where the set of RTS data contains at least one variable that shares the same dynamics origin as that of the TTS, is most appropriate. Such an association will theoretically guarantee that those RTS features, which characterize the intrinsic properties of the system, can identify the most appropriate model from the database for simulating TTS.

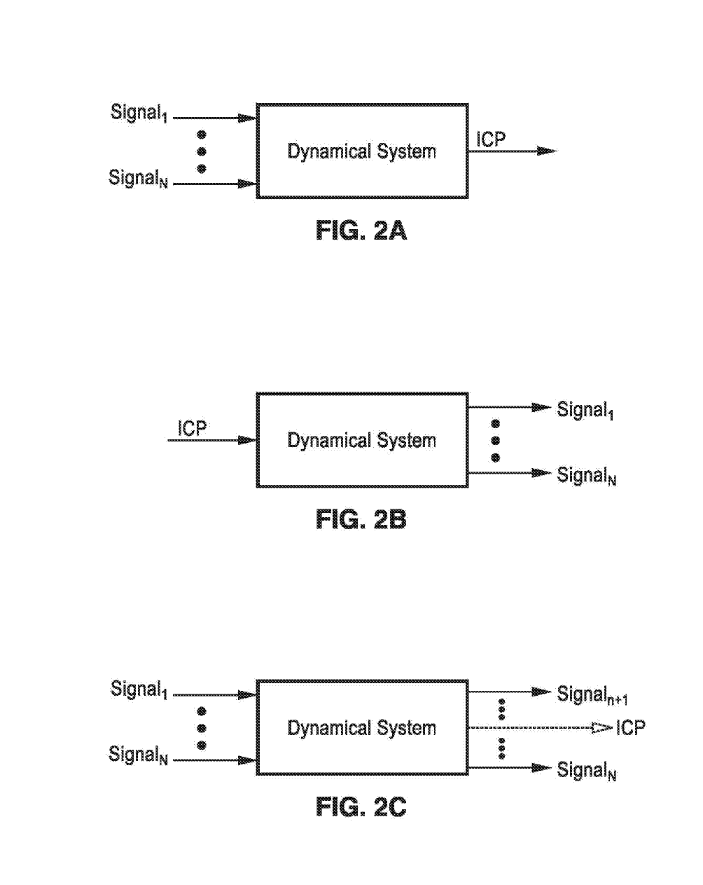

Similarly, there are three possible forms of relationship between ICP and its related noninvasive signals as illustrated in FIG. 2A through FIG. 2C, where Signal; is referred to as a noninvasive ICP-related signal, and it is assumed that there are N of them (N>1). In the first type illustrated in FIG. 2A, ICP is an output of a dynamical system where all the related signals are considered as inputs. In this case, if an accurate model of this system is applicable across different patients, then NICP can be achieved using these input signals to simulate the model. However, it has been shown that such a patient-independent model does not exist to model ABP/CBFV and ICP and is not suitable as a consequence.

In the second type shown in FIG. 2B, ICP assumes an input-signal role and the related signals are outputs. Estimate of input in this scenario can usually be approached using a blind-source identification approach. However, it is not physiologically plausible to treat ICP as input and ABP/CBFV as output of the system. Therefore, this model would be rejected.

The third type, shown in FIG. 2C, describes ICP as among the output signals of a dynamical system and its related signals include both input and output from this dynamical system. For example, the ABP is the input signal to the coupled CBF and CSF circulatory dynamical system from which both ICP and CBFV are output signals.

At block 18 of FIG. 1, simulation models are preferably formulated for each entry in the signal database. In one embodiment, a simulation model may be created by a simulation modeler. In another embodiment, the simulation modeler builds a model directly from a given set of input output data. There are many different types of dynamic models that are available for use by the simulation modeler such as discrete-time, continuous-time, linear and nonlinear models. For example, a stable deterministic linear dynamic model can be used to represent the input/output relationship between ABP, CBFV and ICP in simulations. In other embodiments, a nonlinear dynamic model may be used instead of a linear model. The simulation models formulated at block 18 are typically stored in a database.

At block 20, a metric of dissimilarity is derived through mapping function training. Error exists between the measured output variables and the simulation from the input/output model. The dissimilarity calculations quantify the error. The objective of training is to associate a mapping function with each database entry.

This mapping function takes an available feature vector as input and outputs a dissimilarity measure that predicts how well the input/output model built from this entry will perform in estimating an unavailable variable from the available variable features that is the input to the mapping function. For example, a mapping function can be formulated for each TTS-RTS pair in the database that translates a feature vector extracted from RTS to the dissimilarity between true TTS and its estimate from the dynamic model associated with the same TTS-RTS pair. The association of a mapping function with each database entry can provide an estimate of how well the input/output model derived from this database entry would perform for a given RTS.

The training process at block 20 preferably uses a dissimilarity matrix. The composition of this dissimilarity matrix for a hypothetical N-entry database is one where the kth database entry is associated with an N-dimension column vector that is composed of the dissimilarity measures between each of N variables and their simulated counterparts from using the input/output model associated with the kth database entry.

The dissimilarity matrix may be produced with a feature matrix subject to N×N simulations and evaluations where the rows describe how well the final variable (e.g. ICP) is estimated by models from other database entries and the columns describe how well a model estimates the final variable from other database entries. A linear mapping function is preferably used, incorporating multiple independent regressions to evaluate the entries of the feature matrix. In order to avoid the loss of row wise ranking information, the mapping function regression is preferably constrained and applied iteratively. Up to 2N restraints may be used for each mapping function. In another embodiment, a non-linear kernel function is used as a replacement for certain inner products among various feature vectors.

Using the mapping functions determined in the training process, for example, a query engine can be utilized to select appropriate database entries so that the associated input/output models can be used to predict inaccessible variables with the knowledge of accessible new biological signal variables.

Once the system is trained, it can be used to estimate an unavailable system variable from a new set of available variables at block 22. For example, at run time, a feature vector is extracted from new RTS data and supplied to the mapping function associated with each TTS-RTS pair to calculate a dissimilarity measure. An optimal TTS-RTS pair is then selected by analyzing these dissimilarity measures. The associated input/output model of the selected TTS-RTS pair is then used to simulate the TTS given the inquiry RTS as an input.

Similarly, in the ICP example, a query feature may be extracted from new APB or CBFV or other biological signal data and evaluated by the mapping functions to produce dissimilarity metrics that are ranked to find the optimal model. The selected model is used to simulate the ICP from the new biological signals.

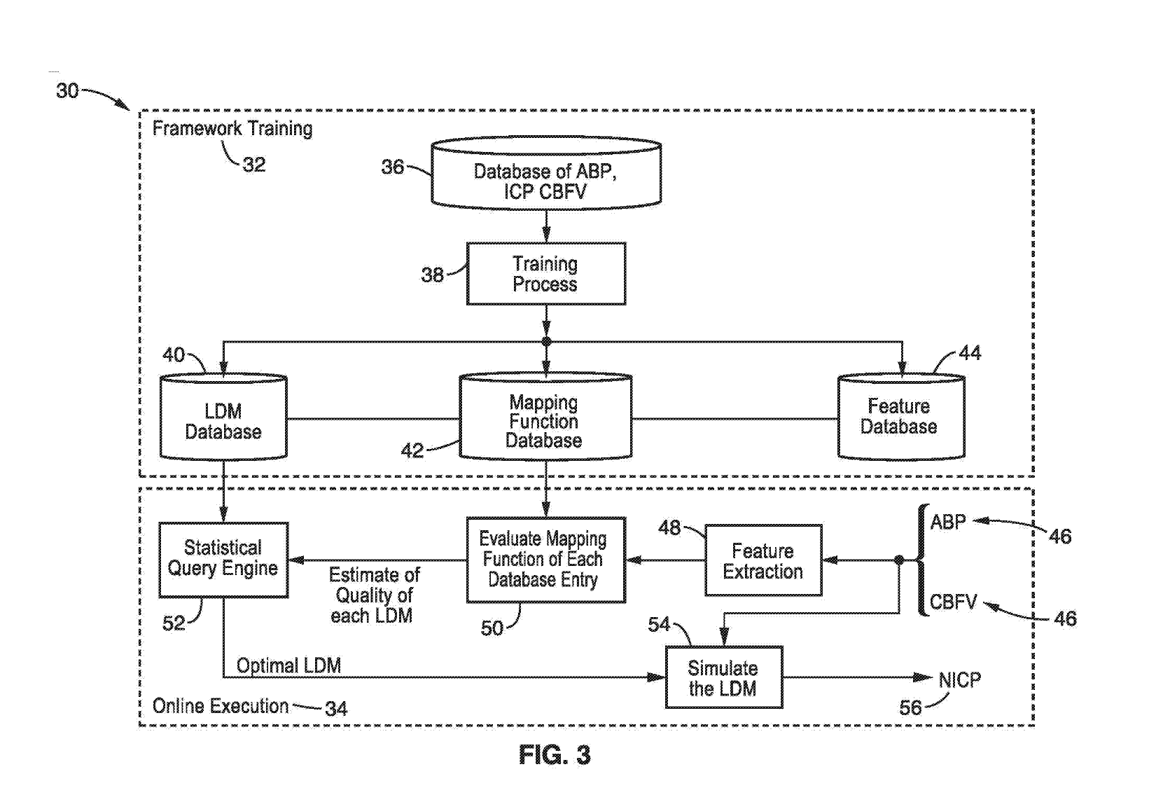

Turning now to FIG. 3, the schematic diagram of one embodiment 30 of the Noninvasive ICP Calibration Framework (NICF) shows a highly modularized framework. The ICP is among the observables of a dynamical system that also includes its related noninvasive signals. The related noninvasive signals can include both input and output from the dynamical system. The operating principle of this NICF framework is to first build a collection of dynamical models and then use new ICP-related signals to locate the optimal models within the collection to simulate ICP for the new signals. As one illustration of this concept, arterial blood pressure (ABP) is the input signal to the coupled CBF and CSF circulatory dynamical system from which both ICP and cerebral blood flow velocity (CBFV) are output signals.

The NCIF framework 30 can be generally divided into an offline framework training module 32 and an online execution module 34. The framework training module 32 orients the selected system from a database of collected biological signal variables as well as dynamic models and simulation models of system and subsystem relationships between the identified variables. Framework training has the goal of associating each database entry with a mapping function.

The online execution module 34 allows the estimation of the desired final variable from new signal variables through the use of the trained framework.

As seen in FIG. 3, the framework training module 32 uses a training process 38 where a database 36 of many instances of invasive ICP, ABP, and CBFV as well as other biological signals are analyzed. In this embodiment, the training 38 results in three types of databases: a database 40 of linear dynamical models (LDM); a database 42 of mapping functions, and a database 44 of query features. The dynamical model database 40 contains the input/output (I/O) models relating ABP/CBFV to ICP that are built from each instance of ABP/CBFV/ICP in this illustration. The mapping function database 42 contains a mapping function for each entry in the model database 40, which provides an estimate of the dissimilarity between the unknown ICP and the NICP simulated using the corresponding dynamical model on a given instance of ABP/CBFV. Each query feature in the feature database 44 is a vector extracted from an instance of ABP/CBFV.

In the online execution module 34 of FIG. 3, a new instance 46 of ABP/CBFV or other variable is acquired and becomes available for analysis. These signals 46 are first analyzed to extract a query feature at block 48 that is further evaluated by each mapping function in the database 42 at block 50. The outputs of this process are the estimated dissimilarity metrics providing an estimate of the quality of the simulated ICP using the models in the database for this given ABP/CBFV instance. These dissimilarity metrics are then ranked to find the optimal model by a statistical query engine at block 52. The returned optimum model is then used to simulate ICP at block 54 using the new ABP/CBFV instance 46 to obtain the noninvasive ICP (NICP) 56 estimate.

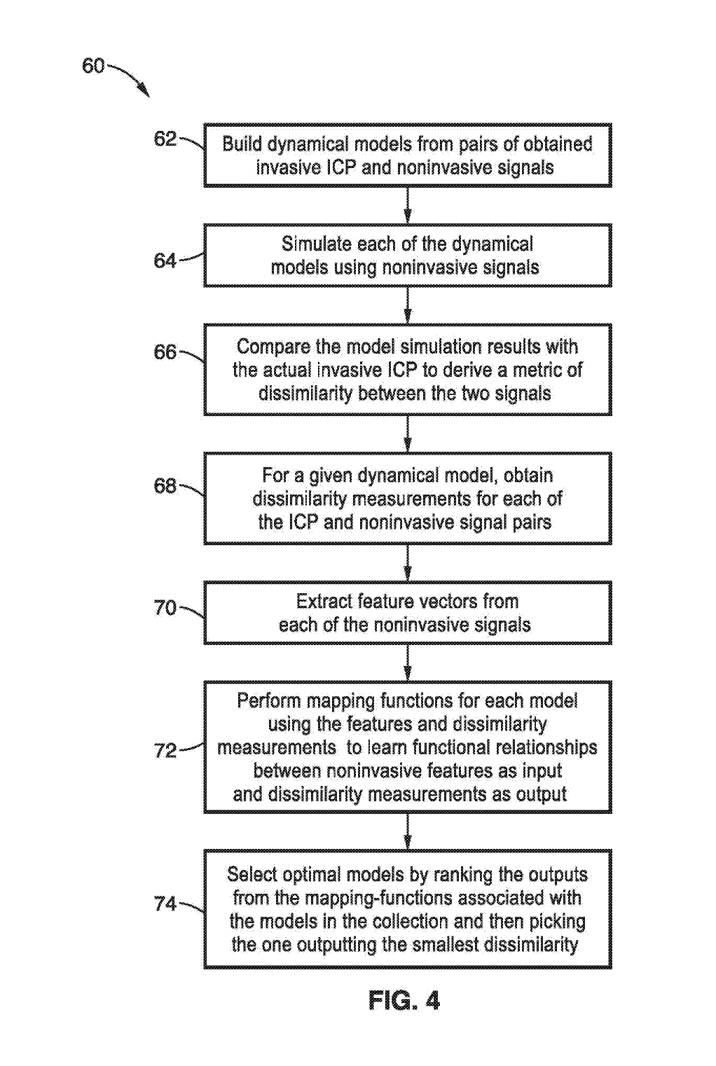

Referring also to FIG. 4, one embodiment 60 of the NCIF algorithm mapping function and selection features is generally described. At block 62 a number N of dynamical models have been built from N pairs of invasive ICP and noninvasive signals as an illustration. At block 64 each of the N models are simulated using N noninvasive signals that have been previously acquired. It is then possible to compare the model simulation results at block 64 with the actual invasive ICP results to derive a metric of dissimilarity between the two signals at block 66. For a given dynamical model, N dissimilarity measurements are obtained for each of the N ICP and noninvasive signal pairs at block 68.

At block 70, N feature vectors are extracted from each of the N noninvasive signals. Using these N features and N dissimilarity measurements, a functional relationship can be learned between a noninvasive feature as input and a dissimilarity measurement as output at block 72. This relationship is called a mapping-function. Obviously, each model will be associated with a mapping-function. This mapping-function essentially provides an estimate of the quality of noninvasive ICP that is obtained by simulating this model. Therefore, an optimal model can be selected at block 74 by ranking the outputs from the mapping-functions associated with the models in the collection and then picking the one outputting the smallest dissimilarity. In addition to estimating ICP, this approach is able to tell users an estimate of how accurate the simulated ICP is and thus can flag a potentially inaccurate noninvasive ICP estimate if the pre-constructed database of models cannot provide an ICP simulation model that is good for the current subject.

The NICF method is also capable of handling any existing noninvasive signals proposed for ICP estimation whose dynamical relationship with an ICP signal can be identified using system identification methods. For example, the approach was implemented and validated on brain injury patients whose invasive ICP was available for both building and validating the approach. The noninvasive signals used in this implementation include cerebral blood flow velocity (CBFV) signals measured at the middle cerebral artery (MCA) using conventional Transcranial Doppler and arterial blood pressure (ABP) signals measured either invasively or noninvasively using Finapres.

The present method is fundamentally different from previous efforts that attempted to use the pulsatility index (PI) of CBFV pulses and have shown conflicting results on estimating ICP. The method utilizes a comprehensive characterization of CBFV pulse morphology to calculate query features for mapping function.

In addition, the performance of detecting intracranial hypertension using pulse morphological features of CBFV pulses was compared with just using the pulsatility index PI alone. It was found that the CBFV pulse morphological features achieved a much higher performance over PI.

The mapping-function learning algorithm illustrated generally in FIG. 1 through FIG. 4 integrates system identification and case based reasoning. Specifically, the algorithm starts with a database with N databases of available biological signal entries such as invasive ICP and noninvasive signals. In learning the mapping function, N×N simulation evaluations are conducted first where each of N models is used to simulate the ICP for each of the N pairs.

By comparing simulated ICP with measured ICP, an N×N dissimilarity matrix E is obtained. The matrix E is organized such that the i-th row represents dissimilarity for the i-th ICP using the N models. Furthermore, the i-th column represents dissimilarity from using the i-th model to simulate ICP for the N entries. A mapping function for the i-th model can be defined as: e=Gᵢ(f) such that e_{j,i}=Gᵢ(fⱼ) where e_{j,i} is an element of E and fⱼ is the feature vector of the j-th entry.

A linear mapping function is used in the form of a naive learning approach formulated as solving N independent linear least squares:

$${min}_{{b}_{i}} {‖ {Fb}_{i} - {e}_{: , i} ‖}_{2}^{2} \qquad i = 1 , 2 , LN$$

where the i-th row of feature F matrix is the feature vector from the i-th entry and bᵢ is a column vector representing the i-th mapping function.

However, this learning approach misses an important piece of information, i.e., the rankings among each row of the matrix E. To demonstrate the importance of ranking constraints, it was assumed that a feature for a new feature is f. To find the optimal linear dynamical model (LDM) for this f, the dissimilarity is calculated from each mapping function so that f^{T}b_{i,} i=1,2, . . . , N is obtained. It is clear now that the correct ranking among f^{T}bᵢ is important to locate the best model for this new f.

To incorporate the row-wise constraints in learning the mapping function, the following least squares problem with constraints can be solved:

$${min}_{{b}_{i}} {‖ {Fb}_{i} - {e}_{: , i} ‖}_{2}^{2} + λ {‖ ζ ‖}_{2}^{2}$$

$$subject to ζ \geq 0$$

$${α}_{i}^{T} FB {β}_{i} \leq {ζ}_{i} , \qquad i = 1 , 2 , {LN}^{3}$$

where ζ is a slack variable for relaxing the constraints, αᵢ is a N×1 vector with only one nonzero element equal to 1, βᵢ is a vector with only two nonzero elements equal to 1 and −1, respectively. So αᵢ^{T}FBβᵢ=fⱼ^{T}(bₖ−bₗ expressing one ranking condition for an arbitrary j, k, l depending on the locations of those nonzero elements in αᵢ and βᵢ.

However, the above formulation is computationally prohibitive due to a large number of constraints ˜O(N³). Therefore, an approximate solution may be used. The solution is to sequentially solve the following convex optimization problem, with the following formulation of the problem at step n:

$${min}_{{b}_{i}} {‖ {Fb}_{n} - {e}_{: , n} ‖}_{2}^{2} + λ {‖ ζ ‖}_{2}^{2}$$

$$subject to ζ \geq 0$$

$$\begin{matrix}{f}_{j , :} {b}_{n} < min ( {{e}^{⋒}}_{j , {L}^{-} ( j , n )} ) + {ζ}_{j} , & j = 1 , 2 , LN \\ {f}_{j , :} {b}_{n} > max ( {{e}^{⋒}}_{j , {L}^{+} ( j , n )} ) + {ζ}_{j + N} , & j = 1 , 2 , LN\end{matrix}$$

In this formulation, it is assumed that bᵢ, i=1, 2, . . . , n−1 is estimated and that bₙ is to be solved. The incorporation of ranking constraints is preferably done by using two lists: L⁺ (j,n)={k|e_{j,n}>e_{j,k} & k<n} and L⁻ (j,n)={k|e_{j,n}<e_{j,k} & k<n} to find the upper and lower bounds for fⱼ:bₙ based on the estimated bᵢ up to n−1. Therefore, the maximal number of constraints is 2N at each step. Conflicts may occur when upper bound is lower than lower bound, however. In such a case, only one of the constraints can be kept so that the actual number of constraints will be smaller than 2N.

To further enhance the flexibility of the mapping function, it will be seen that the above linear solution can be expressed solely in the form of inner products among various feature vectors and hence a “kernel trick” can be played to replace those inner products with a nonlinear kernel function. In this way, the most flexible solution can be obtained that also enjoys the ranking constraints.

In the preceding derivation, the following is a re-formulation of the problem in standard form by removing the subscripts from bₙ and the introduction of a regularization term to handle the under-determined problem when the dimension of the feature space becomes infinite after kernelization:

$${min}_{{b}_{i}} {‖ Fb - e ‖}_{2}^{2} + λ {‖ ζ ‖}_{2}^{2} + y {‖ b ‖}_{2}^{2}$$

$$subject to ζ \geq 0$$

$${f}_{j , :} b \leq {ω}_{j} + {ζ}_{j}$$

Since the solution should be in the row space of F, i.e., b=F^{T}u because the objective function contains only the quadratic terms of b, the following is obtained:

$${min}_{{b}_{i}} {‖ {FF}^{T} u - e ‖}_{2}^{2} + λ {‖ ζ ‖}_{2}^{2} + {yu}^{T} {FF}^{T} u$$

$$subject to ζ \geq 0$$

$${f}_{j , :} {F}^{T} u \leq {ω}_{j} + {ζ}_{j}$$

Then, the substitution of x=FF^{T}u provides:

$${min}_{{b}_{i}} {‖ x - e ‖}_{2}^{2} + λ {‖ ζ ‖}_{2}^{2} + {{yx}^{T} ( {FF}^{T} )}^{1} x$$

$$subject to ζ \geq 0$$

$${x}_{j} \leq {ω}_{j} + {ζ}_{j}$$

It is possible to solve for x and then obtain b by invoking b=F^{T} (FF^{T})¹x. Hence, an estimate of dissimilarity for a new feature vector f_{N+1} can be obtained as e_{N+1}=fₙ₊₁^{T}F^{T} (FF^{T})¹x where only inner products among rows of F and between rows of F and f_{N+1} are involved. By playing the kernel trick, we have:

$$e ( f ) = {\sum }_{i}^{N} κ ( {f}^{T} , {f}_{i , :} ) {K}_{i , :}^{- 1} x$$

where κ is a chosen kernel function and K⁻¹ is the inverse of kernel matrix of training features.

Accordingly, the incorporation of ranking constraints and the kernelized solution are both important elements in improving the accuracy of noninvasive ICP estimation.

The invention may be better understood with reference to the accompanying examples, which are intended for purposes of illustration only and should not be construed in any sense as limiting the scope of the present invention as defined in the claims appended hereto.

### EXAMPLE 1

In order to demonstrate the functionality of the invention, cerebral blood flow velocity (CBFV) was used as a noninvasive signal and the CBFV pulse morphological features were extracted using a pulse analysis algorithm termed Morphological Clustering and Analysis of Intracranial Pulse (MOCAIP). By using the MOCAIP algorithm to analyze CBFV pulses, a significant correlation between morphological metrics of ICP and CBFV pulses was observed. The MOCAIP algorithm was used to analyze CBFV pulses to derive input features for the mapping function.

The MOCAIP analysis starts by detecting individual pulses from the continuous raw pulsatile signal using an algorithm. Consecutive sequences of these raw pulses within a pre-defined time window are clustered, resulting in dominant ICP pulses that are validated against artifact-free pulses in a library of pulses. Only the legitimate dominant pulses are further processed to detect inflection points and local optima of the pulse as candidate peaks. Finally, the peak designation recognizes up to three peaks, in legitimate pulses, among the candidate peaks. There have been defined 128 MOCAIP metrics that can be calculated based on the identified peak locations. These metrics capture morphology of a pulse and have been shown to contain useful input features to various predictive models.

Using the MOCAIP algorithm, simultaneously recorded CBFV (from the MCA) and ICP from 47 brain injury patients were analyzed and then the inter-subject correlation between these 128×128 pairs of metrics was studied using a statistically robust percentage bend correlation method. It was found that 15.2% of metric-pairs between ICP and CBFV pulses have significant (p<0.01) correlation. Since the CBFV pulse morphological features are informative of ICP state, they were a good input feature for mapping function.

The preliminary assessment of the mapping function learning approach was conducted using a database consisting of simultaneously recorded ICP, ABP, and CBFV signals from 38 TBI, 27 aSAH, and 10 hydrocephalus patients. The age span of the database is from 18 to 89 years old with an average age of 43.9 years old. Each signal entry in the database is 300 heartbeats long. Some patients contribute multiple entries and hence we have in total 169 entries in the database. The distribution of four basic vita signs of the 169 entries in the database was evaluated. It was observed that entries with high ICP were rare.

To visualize the quality of the database, a hypothetical querying algorithm was used that always returns the database entry that is guaranteed to produce a model that achieves n-th lowest percentile dissimilarity among all available models. This is a hypothetical approach because it requires that we know the invasive ICP as ground truth. The best performance of such a querying algorithm is for the algorithm to return the entry with lowest dissimilarity. Graphs of 10 overlapped ICP and estimated ICP traces representing different levels of error across the database using the ideal algorithm were generated. Qualitatively, it was seen that the noninvasive ICP can be adequately estimated for up to 75% of the database entries by the ideal mapping function. By increasing percentile of this guaranteed error, the median error across database entries was calculated and listed in Table 1. The median error from a realistic mapping-function learning algorithm can be compared with entries in this table to see how a particular mapping-function learning algorithm stands in this chart.

The distribution of the dissimilarity as a function of difference of a vital sign belonging to a test entry for which the ICP was estimated and that of a model-supplying entry was investigated. The distribution for four vital signs including mean ICP, mean ABP, mean FV, and heart rate (HR) was evaluated. It was revealing to observe that mean ICP difference between model-supplying entry and that of a test entry has the largest influence as there was a clear ditch of dissimilarity at zero difference. This finding showed that an inadequate number of entries of high ICP will limit the performance of any noninvasive ICP approach based on this database.

Using the current database, significant improvement of the newly developed learning algorithm for the mapping function was demonstrated. Furthermore, existing efforts at developing noninvasive sensors for ICP can significantly benefit from this calibration approach.

### EXAMPLE 2

To evaluate the performance of the calibration methods, a 3-fold cross-validation was used to compare the performance of four learning algorithms including the naïve linear mapping, linear mapping with sequential constraints, nonlinear mapping using a Gaussian kernel, and nonlinear mapping using a Gaussian kernel plus the sequential constraints. In each fold, test entries and training entries were from different patients. Except for the linear mapping function, the other three methods had hyper-parameters that will affect performance.

The median error and the standard deviation of the performances from each method were computed. Median error was preferred because there are extremely large errors for the naïve linear mapping function that inflate the mean error.

In terms of absolute error, a paired-sign test showed that all three new methods achieved significantly (p<0.01) better results than the naive linear mapping. Among the three new methods, the nonlinear kernel with ranking constraints was significantly better than both constrained linear (p=0.005) and nonlinear kernel approach (p=0.015). This result clearly documents the progressive methodological improvement that was achieved. Both nonlinear kernel and ranking constraints are important factors in the new learning strategy.

By cross-referencing the displayed median error values to those listed in Table 1, a more intuitive understanding of the results can be obtained. Table 1 lists the median error for a hypothetical query method that always returns the top n-th performing model-supplying entry to a test case. Therefore, in terms of median error, the mapping function from using a nonlinear constraint learning approach is equivalent to a hypothetical query function that returns model-supplying entry among the top 30% performers.

Scatter plots between the true dissimilarity (y-axis) and the estimated dissimilarity (x-axis) from the nonlinear constraint approach were prepared and evaluated. The scatter plots were generated based on the percentiles of the noninvasive ICP error. It was not surprising that test entries with higher ranking-correlation generally have the smallest error (smaller percentile). Please note that every mapping function has to do well to achieve a high-ranking correlation for a new test entry. Therefore, the good result obtained entry cannot be explained by random luck.

From the discussion above it will be appreciated that the invention can be embodied in various ways, including the following:

1. A method of estimating an unknown physiological value from measurements of associated physiological parameters, comprising: (a) creating a database of measurements of a plurality of physiological parameters; (b) providing a database of linear dynamical input/output models built from pairs of physiological parameter measurements; (c) associating a mapping function with each model in the database of models, said mapping function providing at least one dissimilarity value representing dissimilarity between an actual value or function and an estimated value or function from using the model associated with the mapping function; (d) extracting features from a newly acquired set of measurements of one or more physiological parameters; (e) evaluating extracted features with each associated mapping function model producing dissimilarity metrics for each extracted feature; (f) ranking the dissimilarity metrics to find an optimal model; and (g) using the optimal model and the newly acquired set of measurements of one or more physiological parameters to simulate an unknown physiological value.

2. A method as recited in any previous embodiment, further comprising: compiling a database of query features extracted from the database of measurements of a plurality of physiological parameters.

3. A method as recited in any previous embodiment, further comprising: estimating model quality from said ranked dissimilarity metrics.

4. A method as recited in any previous embodiment, wherein the physiological parameters mapping function comprises a linear mapping function with row wise ranking constraints.

5. A method as recited in any previous embodiment, wherein the mapping function further comprises: replacing inner products with a nonlinear kernel function.

6. A method as recited in any previous embodiment, wherein the database of physiological parameters comprises cerebral blood flow velocity (CBFV) signals, intracranial pressure (ICP) signals and arterial blood pressure (ABP) signals from a plurality of subjects.

7. A method of estimating ICP with non-invasive biological signals, the method comprising: (a) creating a database of measurements of ABP, ICP and CBFP from a plurality of test subjects; (b) creating a database of linear dynamical input/output models relating ABP and CBFV with ICP that are built from entries in the ABP, ICP, CBFP database; (c) creating a mapping function database of simulations of each dynamical model with each ABP/CBFV instance in the measurement database; (d) providing an estimate of dissimilarity between an unknown ICP and each simulation ICP; (e) obtaining ABP and CBFV measurements from a patient; (f) extracting features from the ABP and CBFV measurements; (g) evaluating the mapping function of each entry in the mapping function database to produce dissimilarity metrics; (h) ranking the dissimilarity metrics to find an optimal model; and (i) simulating the ICP from the optimal model to estimate ICP of the patient.

8. A method as recited in any previous embodiment, further comprising: creating a database of query features extracted from the database of measurements of a plurality of physiological parameters.

9. A method as recited in any previous embodiment, further comprising: estimating model quality from said ranked dissimilarity metrics.

10. A method as recited in any previous embodiment, wherein the physiological parameters mapping function comprises a linear mapping function with row wise ranking constraints.

11. A method as recited in any previous embodiment, wherein the mapping function further comprises: replacing inner products with a nonlinear kernel function.

12. A method as recited in any previous embodiment, wherein the mapping function comprises:

$$e ( f ) = {\sum }_{i}^{N} κ ( {f}^{T} , {f}_{i , :} ) {K}_{i , :}^{- 1} x ;$$

where f is a new feature, κ is a chosen kernel function and K⁻¹ is the inverse of a kernel matrix of training features.

13. A system for estimating an unknown physiological value from measurements of associated physiological conditions, comprising: (a) sensors adapted to sense physiological conditions of a patient and produce one or more biological signals; and (b) computer with programming on a computer readable storage medium storing instructions which, when executed on a programmed processor, performs steps comprising: receiving biological signals from the sensors; extracting features from one or more received biological signals; comparing extracted features with entries of a database of mapping functions of linear dynamical models producing dissimilarity metrics for each extracted feature; ranking the dissimilarity metrics to find an optimal model; and using the optimal model and the received set of biological signals to simulate an unknown physiological value.

14. A system as recited in any previous embodiment, the programming further comprising: a framework training module; and an execution module; wherein the framework training module comprises a database of recorded biological signals from many different patients, a database of linear dynamic models and a feature database.

15. A system as recited in any previous embodiment, wherein the database of recorded biological signals comprises cerebral blood flow velocity (CBFV) signals, intracranial pressure (ICP) signals and arterial blood pressure (ABP) signals from a plurality of subjects.

16. A system as recited in any previous embodiment, wherein the mapping function of said programming comprises:

$$e ( f ) = {\sum }_{i}^{N} κ ( {f}^{T} , {f}_{i , :} ) {K}_{i , :}^{- 1} x ;$$

where f is a new feature, κ is a chosen kernel function and K⁻¹ is the inverse of a kernel matrix of training features.

17. A system as recited in any previous embodiment, wherein the dissimilarity metrics are placed in a dissimilarity matrix for ranking.

Embodiments of the present invention may be described with reference to flowchart illustrations of methods and systems according to embodiments of the invention, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1
E Returned/Median Error
E return/Median Error

| Smallest | 10% | 20% | 30% | 40% | 50% | 60% | 70% |
|---|---|---|---|---|---|---|---|
| 1.05 | 2.27 | 3.40 | 4.42 | 5.67 | 7.05 | 8.33 | 9.88 |

We claim:

1. A system for estimating an unknown physiological value from measurements of associated physiological conditions, comprising:
(a) a plurality of sensors adapted to sense physiological conditions of a patient and produce one or more biological signals; and
(b) a computer with programming on a computer readable storage medium storing instructions which, when executed on a programmed processor, perform steps comprising:
(i) receiving biological signals from the sensors;
(ii) extracting features from one or more received biological signals;
(iii) comparing extracted features with entries of a database of mapping functions of linear dynamical models producing dissimilarity metrics for each extracted feature;
(iv) ranking the dissimilarity metrics to find an optimal model; and
(v) using the optimal model and the received set of biological signals to simulate an unknown physiological value.

2. A system as recited in claim 1, said programming further comprising:
a framework training module; and
an execution module;
wherein said framework training module comprises a database of recorded biological signals from many different patients, a database of linear dynamic models and a feature database.

3. A system as recited in claim 2, wherein said database of recorded biological signals comprises cerebral blood flow velocity (CBFV) signals, intracranial pressure (ICP) signals and arterial blood pressure (ABP) signals from a plurality of subjects.

4. A system as recited in claim 1, wherein the mapping function of said programming comprises:
$$e ( f ) = {\sum }_{i}^{N} κ ( {f}^{T} , {f}_{i , :} ) {K}_{i , :}^{- 1} x ;$$
where f is a new feature, κ is a chosen kernel function and K⁻¹ is the inverse of a kernel matrix of training features.

5. A system as recited in claim 1, wherein said dissimilarity metrics are placed in a dissimilarity matrix for ranking.
